SEQUENCE LISTING (1 sequence)

SEQ ID NO: 1 - protein, 42 aa, Homo sapiens
DAEFGHDSGFEVRHQKLVFFAEDVGSKKGAIIGLMVGGVVIA

(19) United States
(12) Patent Application Publication
(10) Pub. No.: US 2015/0065448 A1
(43) Pub. Date: Mar. 5, 2015

(54) METHODS OF TREATING AMYLOIDOSES WITH VITAMIN B12 AND DIAGNOSTIC TEST FOR DETECTING THE PRESENCE OF AMYLOID-BETA PEPTIDES

(71) Applicant: Ewa Bienkiewicz, Tallahassee, FL (US)

(72) Inventor: Ewa Bienkiewicz, Tallahassee, FL (US)

(73) Assignee: THE FLORIDA STATE UNIVERSITY RESEARCH FOUNDATION INC., TALLAHASSEE, FL (US)

(21) Appl. No.: 14/237,216

(22) PCT Filed: Aug. 13, 2012
(86) PCT No.: PCT/US12/50582

§ 371 Date: Feb. 5, 2014

Related U.S. Application Data
(60) Provisional application No. 61/522,759, filed on Aug. 12, 2011.

(51) Int. Cl. A61K 31/714; G01N 33/68

(52) U.S. Cl. CPC A61K 31/714; G01N 33/6896; G01N 2333/47; G01N 2800/2821
USPC 514/52; 436/501

(57) ABSTRACT

Embodiments of the invention include methods of using vitamin B12 to influence amyloid-beta peptide aggregation. A preferred method comprises contacting at least one amyloid-beta peptide oligomer formed from a plurality of amyloid-beta peptide monomers with an effective amount of vitamin B12, the effective amount being sufficient to stimulate dissociation of the amyloid-beta peptide monomers from the amyloid-beta peptide oligomer or fibril. Also provided are methods of treating Alzheimer's disease and diagnostic tests for detecting the presence of amyloid-beta peptides.

### CROSS-REFERENCE TO RELATED APPLICATION

Priority is claimed to U.S. provisional application Ser. No. 61/522,759 filed on Aug. 12, 2011, entitled “Therapeutic Agents for Alzheimer's Disease,” which is incorporated by reference in its entirety.

### FIELD OF THE INVENTION

This invention relates to the field of treatment of amyloidoses associated with amyloid-beta peptides. More particularly, the invention relates to methods of treating amyloid-beta peptide aggregation and diagnostic tests for detecting the presence of amyloid-beta peptides.

### SEQUENCE LISTING

This application contains a Sequence Listing electronically submitted via EFS-web to the United States Patent and Trademark Office as a text file named “Sequence_Listing.txt.” The electronically filed Sequence Listing serves as both the paper copy required by 37 C.F.R. §1.821(c) and the computer readable file required by 37 C.F.R. §1.821(c). The information contained in the Sequence Listing is incorporated herein by reference in its entirety.

### BACKGROUND

Amyloidoses are pathological conditions characterized by the aggregation of certain proteins into harmful oligomers and deposits called amyloid fibrils. These self-associated amyloid species are toxic to various types of cells, including certain type of brain cells. A particularly prominent example of an amyloidosis is Alzheimer's disease. In Alzheimer's disease, amyloid oligomers and fibrils accumulate in the brain, deteriorating the brain's memory functions.

The amyloid oligomers and fibril deposits in the brain of a patient with Alzheimer's disease are formed from a particular peptide called amyloid-beta peptide. Amyloid-beta peptide, which is also referred to “Aβ”, is a protein of about 40-43 amino acid residues. Its 40 amino acid form (Aβ 1-40), 42 amino acid form (Aβ 1-42), and 43 amino acid form (Aβ 1-43) have been associated with amyloid oligomer formation and fibril deposits. Amyloid-beta peptide is the major constituent of neuritic plaque deposits, which are distributed throughout the walls of cerebral blood vessels and the neuropil of the central nervous system.

Approximately five million Americans are currently afflicted with Alzheimer's disease (“AD”), with a new case identified every 70 seconds. The AD diagnosis places each patient on an irrevocable path of fatal neurodegeneration, as there is currently no effective cure for this devastating disease. At present, the treatment options for Alzheimer's disease include cholinesterase inhibitor-type and receptor agonist-type drugs. These drugs help somewhat, but are not fully effective.

### SUMMARY

At the heart of the invention, is my discovery that vitamin B12 prevents individual amyloid-beta peptides from aggregating to form the harmful amyloid oligomers and fibrils and, remarkably, even causes amyloid fibrils that have already formed to dissociate. This has allowed me to develop methods of treating amyloid-beta peptide aggregation and, thereby, treatment methods for Alzheimer's or Alzheimer's-like pathology. These treatment methods are described below.

In a first embodiment of the invention, a method of treating amyloid-beta peptide aggregation in a patient having Alzheimer's disease comprises: determining a first level of amyloid-beta peptides in the patient; establishing a first dose of vitamin B12 based on the first level of amyloid-beta peptides in the patient, the first dose being sufficient to promote dissociation of amyloid-beta peptide monomers from amyloid-beta peptide aggregates in the patient; and administering the first dose to the patient.

In a second embodiment of the invention, a method of treating amyloid-beta peptide aggregation in a subject, comprising contacting at least one amyloid-beta peptide oligomer formed from a plurality of amyloid-beta peptide monomers with an effective amount of vitamin B12, the effective amount being sufficient to stimulate dissociation of the amyloid-beta peptide monomers from the amyloid-beta peptide oligomer.

In a third embodiment of the invention, a method of treating Alzheimer's disease comprises administering an effective amount of vitamin B12 to a patient, the effective amount being sufficient to cause dissociation of amyloid-beta peptide monomers from pre-existing amyloid-beta peptide oligomers in the patient, the effective amount being based on a pre-determined quantity of amyloid-beta peptide oligomers in the patient.

In a fourth embodiment of the invention, a method of preventing neuronal-cell death comprises estimating a level of amyloid-beta peptides in a subject; establishing an effective amount of vitamin B12 based on the estimated level; and administering the effective amount of vitamin B12 to the subject.

A test for detecting the presence of amyloid-beta peptides, the test comprising stimulating a signal from a sample having been exposed to vitamin B12 and identified as being at risk for containing amyloid-beta peptides; and detecting the signal, the signal being indicative of the presence or absence of amyloid-beta peptides.

These and other aspects, embodiments, and features of the invention will be better understood in the context of the accompanying drawings and the following Detailed Description of Preferred Embodiments.

### BRIEF DESCRIPTION OF THE DRAWINGS

FIG. 1 is a diagram illustrating the location, sequence, and structure of the amyloid-beta peptide along the β-amyloid precursor protein (PAPP);

FIG. 2 is a diagram illustrating the various degrees of agglomeration of amyloid-beta peptide from monomer to plaque;

FIG. 3 is a graph showing a plurality of intrinsic tyrosine fluorescence spectra of fresh amyloid-beta peptide in the presence of different concentrations of vitamin B12;

FIG. 4 is a graph of the change in the fluorescence intensity of the data in FIG. 3 as a function of the concentration of vitamin B12;

FIG. 5 is a graph showing a plurality of intrinsic tyrosine fluorescence spectra of aged amyloid-beta peptide in the presence of different concentrations of vitamin B12;

FIG. 6 is a graph of the change in the fluorescence intensity of the data in FIG. 5 as a function of the concentration of vitamin B12;

FIG. 7 is a surface plasmon resonance (SPR) kinetic sensorgram of amyloid-beta in the presence of different concentrations of vitamin B12;

FIG. 8 is calculated fit of the SPR data of FIG. 7 using a 1:1 (B12:Aβ) binding model;

FIG. 9 is a graph of the fluorescence intensity of Thioflavin T in samples containing amyloid-beta peptide and vitamin B12, in which the amyloid-beta peptide was incubated in the presence of vitamin B12(▪), absence of vitamin B12 (♦), and in which the amyloid fibrils formed in the experiments represented by ♦ were subsequently treated with vitamin B12 (▴);

FIG. 10 is a graph of the lag phase of the samples of FIG. 9;

FIG. 11 is a series of micrographs of the samples indicated; and

FIG. 12 is bar graph showing the quantification of cell death counts for the samples represented in FIG. 11, in which the numbers in parenthesis reflect the number of trials and the numbers in brackets represent the Aβ:B12 ratio.

### DETAILED DESCRIPTION OF PREFERRED EMBODIMENTS

In the Summary above and in the Detailed Description of Preferred Embodiments, reference is made to particular features (including method steps) of the invention. Where a particular feature is disclosed in the context of a particular aspect or embodiment of the invention, that feature can also be used, to the extent possible, in combination with and/or in the context of other particular aspects and embodiments of the invention, and in the invention generally.

The term “comprises” is used herein to mean that other features, ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

This invention may be embodied in many different forms and should not be construed as limited to the embodiments set forth herein. Rather, these embodiments are provided so that this disclosure will convey preferred embodiments of the invention to those skilled in the art.

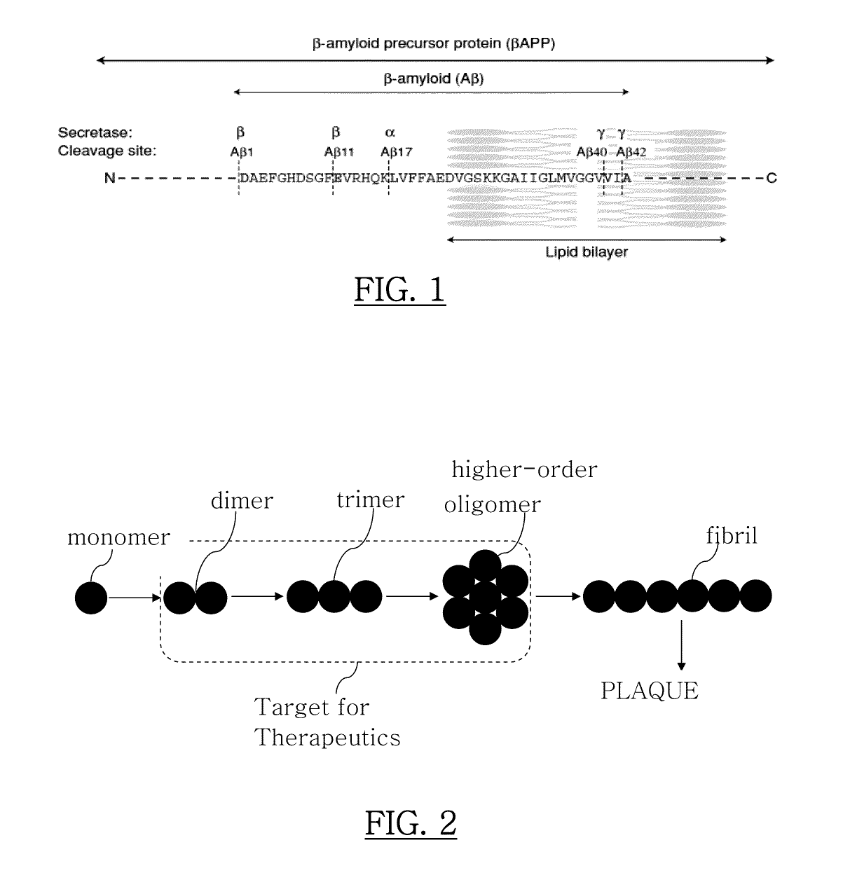

As mentioned above, one of the key components to blame for the pathology of Alzheimer's disease is the amyloid-beta (Aβ) peptide. Referring to FIG. 1, the amyloid-beta peptide is a fragment of the beta-amyloid precursor protein (βAPP). When βAPP is cleaved, it releases the 40-42 amino acid fragment amyloid-beta peptide.

With reference now to FIG. 2, amyloid-beta peptide monomers have an ability to self-associate or aggregate into higher-order oligomeric structures (dimers, trimers, etc.), fibrils, and plaques. Recent evidence shows that the amyloid-beta peptide's structural diversity translates into variations in its neurotoxicity, with the dimeric/oligomeric states shown to be the most harmful, and the monomeric form thought to be neuroprotective.

Vitamin B deficiency has been implicated in processes affecting the progression of Alzheimer's-like pathology through (1) abnormal phosphorylation of the tau protein and formation of neurofibrillary tangles (Nicolia, et al, Journal of Alzheimers Disease, Vol. 19(3), pages 895-907 (2010)), (2) increased production of the amyloid-beta peptide (Fuso, et al, Molecular and Cellular Neuroscience, Vol. 37(4), pages 731-746 (2008)), and (3) increased self-association of the amyloid-β peptide leading to neurotoxicity (described here). It is plausible that the therapeutic effects of vitamin B12 for AD and AD-like pathologies may include these three pathways in concert.

Although the mechanisms underlying these phenomena require further investigation, it is clear that any factors that modify amyloid-beta peptide self-association are likely to influence the survival of neuronal cells. In this context, I hypothesized that directly binding small molecules to the various structural forms of the amyloid-beta peptide may be an effective way shift the relative distribution of the amyloid-beta structures away from the toxic oligomers, fibrils, and plaques toward the monomer form.

Numerous clinical investigations have demonstrated that vitamin B12 is crucial for maintaining cognitive function. Patients exhibiting Alzheimer's-like pathology display a marked decrease in vitamin B12 levels. This observation implies a “loss of function” scenario, where tasks normally carried out by vitamin B12 are attenuated. Although vitamin B12 is known for its function as a co-factor in metabolic reactions and DNA synthesis, I hypothesized that vitamin B12 may also play a direct role in modulating the levels of toxic amyloid-beta aggregates. As a “small molecule”, I suspected that vitamin B12 could carry out this task by directly interacting with the amyloid-beta peptide, thereby modulating its ability to self-assemble and reducing its toxicity. The experimental results presented here demonstrate that vitamin B12 influences both the conformational state adopted by the amyloid-beta peptide and the extent of neuronal cell death triggered by its presence.

Using a multidisciplinary approach, combining biophysical and neuronal-cell culture studies, I show that vitamin B12 is a neuroprotective binding partner of the amyloid-beta peptide. This indicates that B12 plays a preventive role in the process of self-association and formation of toxic amyloid-beta peptide aggregates that have been demonstrated to be an integral part of the AD pathology. My findings are consistent with clinical observations where diminished levels of vitamin B12 were observed to accompany the progression of Alzheimer's disease. They also point to vitamin B12's utility as a therapeutic agent that alleviates neuronal-cell death by modulating Aβ peptide aggregation and formation of toxic species. The efficacy of treatment by vitamin B12 may be enhanced by utilizing targeted delivery of vitamin B12 to the brain and/or by derivatization of the vitamin B12 molecule.

At the outset, it should be understood that the term “vitamin B12” as used herein refers to the compound cobalamin and its natural and synthetic derivatives, which include, but are not limited to, cyanocobalamin, methylcobalamin, adenosylcobalamin, and hydroxycobalamin.

As briefly mentioned in the Summary section above, preferred embodiments of the invention include various methods of influencing amyloid-peptide aggregation using vitamin B12. Those preferred embodiments will now be described in more detail.

Remarkably, the results discussed in the Examples section show that vitamin B12 causes amyloid-beta peptide monomers to dissociate from the higher-order structural form of amyloid-beta peptides. These higher-order structural forms (dimers, trimers, higher-order oligomers, fibrils, etc.) are referred to herein as amyloid-beta peptide aggregates because they contain a plurality of aggregated amyloid-beta peptide monomers.

In a first embodiment of the invention, a method of treating amyloid-beta peptide aggregation in a patient having Alzheimer's disease comprises determining a first level of amyloid-beta peptides in the patient, establishing a first dose of vitamin B12 based on the first level of amyloid-beta peptides in the patient, the first dose being sufficient to promote dissociation of amyloid-beta peptide monomers from the amyloid-beta peptide aggregates in the patient, and administering the first dose to the patient.

Optionally, this method forms a part of a treatment regimen in which the steps are repeated multiple times and the dosage of vitamin B12 in each subsequent dose is adjusted according to the level of amyloid-beta peptide aggregates remaining in the patient after the previous dose. In this case, the method further comprises determining a second level of amyloid-beta peptides in the patient, establishing a second dose of vitamin B12 based on the second level of amyloid-beta peptides in the patient, and administering the second dose to the patient.

The level of amyloid-beta peptides in the patient is preferably determined by extracting a plasma sample from the patient and measuring the quantity of amyloid-beta peptide in the sample. Suitable techniques for measuring the quantity of amyloid-beta peptides in the sample already exist. Some examples of these techniques are now described.

One preferred technique for measuring the quantity of amyloid-beta peptide in a sample employs a sandwich Enzyme-Linked Immunosorbent Assay, or ELISA, methodology. Typically, ELISA utilizes a system with two antibodies. One is immobilized on a surface (capture antibody) and serves to recognize and bind an antigen, in this case the amyloid-beta peptide. The second antibody (detection antibody) recognizes the same antigen (the amyloid-beta peptide) and is conjugated to a system that allows for detection (signal amplification) of the original capture antibody/amyloid-beta peptide complex. An example of the detection system is horse radish peroxidase (HRP) and alkaline phosphatase (ALP), where an enzymatic reaction leads to a change in color (detection/signal amplification) in the presence of the antigen-antibody complex. In a standard protocol, the major steps include: (1) application of sample onto a surface with the capture antibody, (2) washing off the unbound material, (3) applying the second (detection) antibody, (4) washing off unbound material, and (5) executing of a reaction/protocol that allows for detection and quantitation of the antigen. A standard curve generated using known concentrations of the antigen (amyloid-beta peptide) is used to interpolate the concentration of the antigen of interest in the respective sample. ELISA kits for detection of various isoforms of the amyloid-beta peptide are available from several vendors. Examples include: BETAMARK® Beta-Amyloid x-42; Chemiluminescent ELISA Kit, Covance, Catalog Number: SIG-38952 (detects amyloid-beta x-42); BETAMARK® Total Beta-Amyloid; Chemiluminescent ELISA Kit, Covance, Catalog Number: SIG-38966 (detects amyloid-beta 1-38, 1-40, 1-42, and 1-46 of amyloid-beta peptide; Human Amyloidβ(1-x) Assay Kits, Immuno-Biological Laboratories Co., Ltd. (detect Aβ(1-40), Aβ(1-42) and Aβ(N3pE-42) separately in plasma, cerebrospinal fluids, and serum); and ABtest for quantitative determination of beta-amyloid pool in blood, Araclon Biotech, (detects 1-40 and 1-42 isoforms of amyloid-beta found in free plasma and bound to other plasma components, including lipids and proteins, and to blood cells).

An alternative method, still in development, for measuring the quantity of amyloid-beta peptide in a sample is based on two-dimensional infrared (2D IR) spectroscopy analysis of white blood cells (or mononuclear leukocytes). This method measures enrichment in the β-sheet secondary structure and increase in carbonyl signal resulting from accumulation of the amyloid beta species and progression of the Alzheimer's disease. This technique is described by Carmona, et al in Analytical and Bioanalytical Chemistry, Vol. 402(6): pages 2015-2021 (2012). The portion of this references describing the 2D IR technique is incorporated by reference herein in its entirety.

The amount of vitamin B12 in a particular dose is determined based on the level of amyloid-beta peptides in the patient. The hippocampal cell culture results show that, when the ratio of vitamin B12 to amyloid-beta peptide is approximately 1:1, optimum effectiveness is achieved. Administering an excess of vitamin B12 does not appear to enhance the amount of amyloid-beta peptide monomers dissociated from the amyloid-beta peptide aggregates, but administering less vitamin B12 diminishes the amount of amyloid-beta peptide monomers dissociated from the amyloid-beta peptide aggregates. Accordingly, the dose of vitamin B12 is preferably at least equimolar with the molarity of amyloid-beta peptide in the patient estimated from the plasma sample. In the treatment regimen, the amount of vitamin B12 in each subsequent dose is adjusted according to the level of amyloid-beta peptide aggregates estimated to be in the patient after a previous dose.

There are many conventional techniques for administering a pharmaceutical composition to patient, which may also be used to administer vitamin B12 to the patient. These administration techniques include, but are not limited to administering one or more pharmaceutically acceptable dosage forms such as a suspensions, tablets, suppositories, capsules, injectables, transdermals or the like that can be administered to a human or animal patient. Other suitable administration techniques include oral, sublingual, buccal, intravenous, subcutaneous, transcutaneous, intramuscular, intracutaneous, intrathecal, epidural, intraocular, intracranial, inhalation, intranasal, or the like. Any combination of these administration techniques may also be used.

Optionally, vitamin B12 is an active ingredient in a pharmaceutical composition. In such embodiments, vitamin B12 is blended with one or more ingredients useful for making the composition into a pharmaceutically acceptable dosage form such as a suspension, tablet, capsule, injectable, or the like that can be administered to a human or animal patient. Exemplary ingredients include one or more excipients, diluents, disintegrants, emulsifiers, solvents, processing aids, buffering agents, colorants, flavorings, solvents, coating agents, binders, carriers, glidants, lubricants, granulating agents, gelling agents, polishing agents, suspending agent, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, emollients, dissolution enhancers, dispersing agents, or combinations thereof.

Optionally, vitamin B12 is included in a composition having one or more additional active ingredients that bind to amyloid-beta peptide. Examples of these additional active ingredients include, but are not limited to caffeine, resveratrol, melatonin, vitamin B6, and folic acid.

In a second embodiment of the invention, a method of treating amyloid-beta peptide aggregation in a subject comprises contacting at least one amyloid-beta peptide oligomer formed from a plurality of amyloid-beta peptide monomers with an effective amount of vitamin B12, the effective amount being sufficient to stimulate dissociation of the amyloid-beta peptide monomers from the amyloid-beta peptide oligomer.

The effective amount of vitamin B12 used in this method is a function of the quantity of amyloid-beta peptides in a sample taken from the subject. The sample is preferably a sample of the subject's plasma. The quantity of amyloid-beta peptides in the subject's plasma is preferably determined using one or more of the techniques discussed above.

The effective amount of vitamin B12 is preferably adapted to provide an approximately 1:1 ratio of vitamin B12 to amyloid-beta peptide in the patient. Accordingly, a preferred effective amount is at least equimolar with the molarity of amyloid-beta peptide in the patient, as estimated from the sample. Another preferred effective amount of vitamin B12 is at least one molecule of vitamin B12 per amyloid-beta peptide monomer dissociated from the amyloid-beta peptide oligomer.

The “subject” referred to here is preferably a human or animal subject that has been identified as having a condition characterized by amyloid-beta peptide aggregates, including but not limited to Alzheimer's disease.

The term “contacting” refers to placing vitamin B12 in direct physical association with the subject. This is achieved using either a solid, liquid, or gaseous form of a composition comprising vitamin B12. It includes events that take place both intracellularly and extracellularly and may be accomplished by any of the administration techniques set forth above or any other conventional drug administration technique.

In a third embodiment of the invention, a method of treating Alzheimer's disease comprises administering an effective amount of vitamin B12 to a patient, the effective amount being sufficient to cause dissociation of amyloid-beta peptide monomers from pre-existing amyloid-beta peptide oligomers in the patient, the effective amount being based on a pre-determined quantity of amyloid-beta peptide oligomers in the patient.

As with the embodiments previously described, the quantity of amyloid-beta peptides in the patient is preferably determined from the level of amyloid-beta peptide in a sample of the patient's plasma and is at least an equimolar ratio of vitamin B12 to amyloid-beta peptides in the sample. Vitamin B12 is preferably administered using one or more of the administration techniques discussed above.

In a fourth embodiment of the invention, a method of preventing neuronal-cell death comprises estimating a level of amyloid-beta peptides in a subject, establishing an effective amount of vitamin B12 based on the estimated level, and administering the effective amount of vitamin B12 to the subject. Preferably, the level of amyloid-beta peptides in the subject is estimated by testing the subject's plasma for the quantity of amyloid-beta peptides therein using or more of the previously described techniques. The effective amount of vitamin B12 is preferably at least an equimolar ratio of vitamin B12 to amyloid-beta peptides estimated to be in the subject. This method is particularly useful at preventing the death of hippocampal cells.

The amount or quantity of amyloid-beta peptides in the subject is estimated by determining the quantity of amyloid-beta peptides in the sample and extrapolating to account for the estimated total volume of the fluid tested in the subject. For example, the amount of amyloid-beta peptide in the subject's plasma is estimated by determining the quantity of amyloid-beta peptides in a known volume of the sample and extrapolating this quantity to correspond to the total amount of plasma in the subject. The total amount of plasma in the subject can be estimated from the volume of blood in the subject. The same technique is useful for estimating the amount of amyloid-beta peptides in the other embodiments described herein.

The results discussed in the Examples section show that vitamin B12 influences the way that amyloid-beta peptides respond to external stimuli such as electromagnetic radiation. This is because vitamin B12 inhibits amyloid-beta peptide monomers from self-associating and because vitamin B12 binds directly with the amyloid-beta peptide monomers. Advantageously, vitamin B12's influence on the amyloid-beta peptides can be exploited to provide a diagnostic test for detecting amyloid-beta peptides in a sample containing biological material such as bodily fluid or bodily tissue. Accordingly, another aspect of the invention is a diagnostic test for detecting the presence of amyloid-beta peptides.

A diagnostic test for detecting the presence of amyloid-beta peptides, according to yet another embodiment of the invention comprises stimulating a signal from a sample having been exposed to vitamin B12 and identified as being at risk for containing amyloid-beta peptides and detecting the signal. The signal indicates the presence or absence of amyloid-beta peptides in the sample.

The signal is preferably stimulated and detected using a spectroscopic technique such as fluorescence spectroscopy or surface plasmon resonance (SPR) spectroscopy.

When fluorescence spectroscopy is employed, a fluorescence signal from tyrosine, intrinsic to the amyloid-beta peptides, is detectable. The strength of the fluorescence signal is a function of the concentration of vitamin B12 in proximity to the tyrosine on the amyloid-beta peptides. In general, the strength of the tyrosine fluorescence signal tends to decrease with increasing concentration of vitamin B12.

Alternatively, when fluorescence spectroscopy is employed, the fluorescence signal may be stimulated from a fluorophore associated with the sample. Suitable fluorophores include, but are not limited to, benzothiazole dyes such as thioflavin T (“ThT”).

Some particular embodiments of this diagnostic test allow for a determination of the degree of aggregation among amyloid-beta peptide monomers in the sample. This is achieved by comparing successively detected fluorescence signals stimulated from the fluorophore. In the fluorescence spectroscopy experiments described in the Examples section, I show that successively detected fluorescence signals from samples of fluorophore-associated amyloid-beta peptide reveal whether, and by what extent, the amyloid-beta peptides have aggregated in the sample.

When SPR spectroscopy is employed, the sample is preferably positioned on a substrate and a solution of vitamin B12 is flowed over the substrate. The SPR detected signal is a function of the degree of binding between vitamin B12 and amyloid-beta peptides in the sample.

### EXAMPLES

The embodiments of the invention described above will be even better understood in the context of the following examples. These examples are not intended to limit the scope of the invention in any way.

### Example 1

### Vitamin B12 Binds to Amyloid-Beta Peptide

This section shows that vitamin B12 binds to amyloid-beta peptide.

Preparation of Amyloid-Beta Peptide Samples.

Synthetic Aβ(1-40) peptide was purchased from Biopeptide, Inc. at a purity level ≧98%. The hydroxocobalamin form of vitamin B12 and Thioflavin T were purchased from Sigma-Aldrich. All other reagents were of analytical grade.

Samples of Aβ(1-40) were reconstituted in phosphate buffer saline (PBS) (137 mM NaCl, 2.7 mM KCl, mM Na₂HPO₄, 2 mM KH₂PO₄, pH 7.4), except in surface plasmon resonance experiments, as described below. Immediately after reconstitution into PBS, the pH of the solution was adjusted to 10, allowing for solubilization of the peptide. The pH was then readjusted to 7.4, followed by filtration through a 0.22 μm supor membrane filter. Concentrations of Aβ(1-40) were determined using UV-Vis spectroscopy (Cary 300 spectrophotometer, Varian, Inc) and an extinction coefficient of 1,490 M⁻¹cm⁻¹ at 280 nm. Aβ(1-40) samples used in fluorescence experiments to examine peptide/B12 binding included freshly prepared, monomeric Aβ species and “aged” Aβ, incubated for seven days and containing high-molecular weight species.

Preparation of Vitamin B12 Samples.

Hydroxocobalamin form of the vitamin B12 was dissolved in phosphate buffer saline (PBS), pH 7.4, unless stated otherwise, followed by filtration through a 0.22 μm supor membrane filter. The vitamin B12 samples were used within twenty four hours of preparation, stored protected from light. Concentrations of B12 were determined using UV-Vis spectroscopy (Cary300 spectrophotometer, Varian, Inc) and an extinction coefficient of 4,900 M⁻¹ cm⁻¹ at 525 nm.

Intrinsic Tyrosine Fluorescence Spectroscopy Experiments on Amyloid-Beta Peptide in the Presence of Vitamin B12.

Intrinsic tyrosine fluorescence was measured as previously described by Lakowicz in Principles of Fluorescence Spectroscopy. 2 ed. Vol. 1. 2004, New York: Springer. 445-486, which is hereby incorporated by reference in its entirety. Fluorescence emission spectra of the Aβ(1-40) peptide at 50 μM in PBS were recorded at 25° C. using a Cary Eclipse spectrometer equipped with Peltier temperature control (Varian, Inc). All solutions were filtered through 0.22 μm supor membrane filters. Aβ(1-40) samples containing varying amounts of B12 were excited at 265 nm. The emission spectra were collected at 285-370 nm wavelength range, with each spectrum was corrected for buffer contribution. The intensity for each spectrum was normalized to the highest value detected in the absence of vitamin B12 and plotted as a function of the change in fluorescence intensity. The binding constant, K_{D}, was calculated using equation (1) in SigmaPlot 9.0 (Systat Software, Inc.) assuming 1:1 binding stoichiometry.

$$f = \frac{{B}_{max} * abs ( x )}{{K}_{D} + abs ( x )} \qquad ( 1 )$$

Static Vs. Dynamic Quenching.

All experiments were performed at 25° C., unless otherwise stated. Absorption spectra were recorded using a UV-Vis Cary300 spectrophotometer (Varian, Inc.) Fluorescence spectra were normalized and corrected for wavelength-dependent sensitivity. Dynamic or collisional quenching was investigated using the Stern-Volmer equation:

$$\frac{{F}_{0}}{F} = 1 + {k}_{q} {τ}_{0} [ Q ] = 1 + {K}_{D} [ Q ] \qquad ( 2 )$$

where F₀ and F are the fluorescence intensities in the absence or presence of the quencher, B12, respectively; k_{q} is the biomolecular quenching constant; τ₀ is the lifetime of the unquenched tyrosine (3.2 ns) [34], [Q] is the concentration of B12. The Stern-Volmer quenching constant is given by K_{D}=k_{q}τ₀.

Surface Plasmon Resonance (SPR) Experiments.

The binding of Aβ(1-40) and vitamin B12 was analyzed by SPR using a Biacore T-200 optical biosensor (Biacore/GE Healthcare). The Aβ peptide was immobilized on CM5 sensor chips using amine coupling chemistry [36]. Briefly, the carboxymethyl dextran surface of a CM5 sensor chip was activated with a 1:1 mixture of 0.4 M 1-ethyl-3-(3-dimethylaminopropyl)-carbodiimide (EDC) and 0.1 M N-hydroxysuccinimide (NHS) at 25° C. A peptide surface (400-800 RU) was created by pulsing Aβ in 10 mM sodium acetate at pH 4.5 over the flow cell. The remaining binding sites on the dextran surface were blocked with 1.0 ethanolamine-HCl, pH 8.5. A reference surface was activated and blocked as outlined above.

Binding experiments were carried out using HBS-EP+ running buffer (0.1 M Hepes, 1.5 M NaCl, 30 mM EDTA, 0.05% v/v surfactant P-20, pH 7.4) at 25° C. All components were 0.22 μm-filtered prior to use. For analysis of kinetic constants, varying concentrations of vitamin B12, ranging from 0 to 1000 μM, were injected over the chip surface at 75 μL/min. Each analyte concentration was injected in triplicate. Surface regeneration was achieved with 10 mM glycine, pH 2.5 and 0.05% SDS at a flow rate of 50 μL/min. Data were analyzed using T200 BiaEvaluation 1.0 (Biacore) software and fit to a 1:1 binding model.

Results.

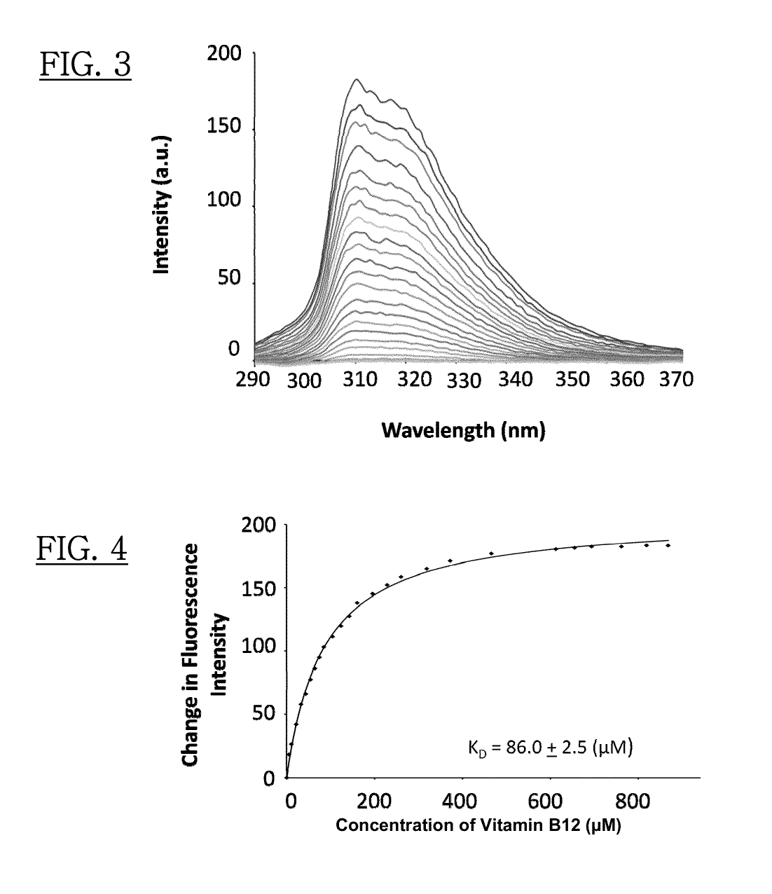

Binding of B12 to the Aβ peptide was demonstrated using fluorescence and surface plasmon resonance (SPR). Fluorescence experiments were carried out by measuring changes in the emission signal of Aβ(1-40), with its tyrosine residue serving as an intrinsic probe (Ex=265 nm). Vitamin B12 was titrated stepwise into the freshly prepared and aged Aβ(1-40) samples, respectively (FIGS. 3 and 4), providing a way calculate the binding constants and to identify any differences in vitamin B12's affinity for the monomeric (freshly prepared) and self-associated (aged, or higher-order) Aβ forms.

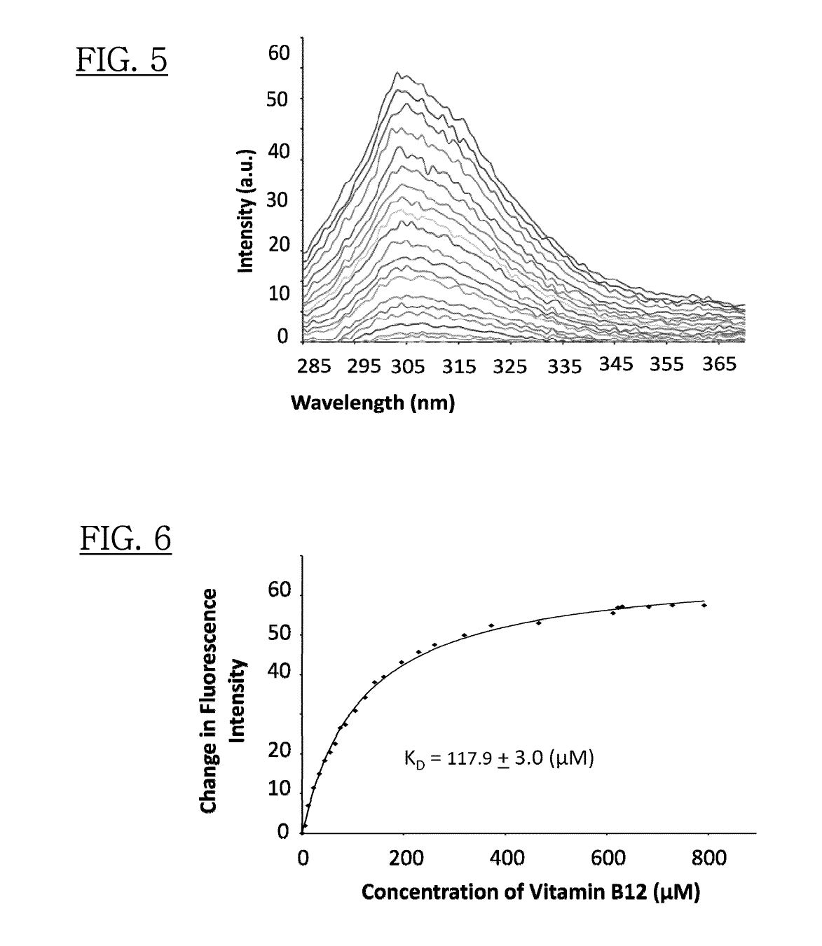

In FIGS. 5 and 6 the change in fluorescence signal at 305 nm (signal max) upon addition of vitamin B12 is plotted as a function of varying B12 concentration. The resulting binding constants were 87.0±2.7 μM for monomeric and 96.1±3.5 μM for self-associated Aβ peptide.

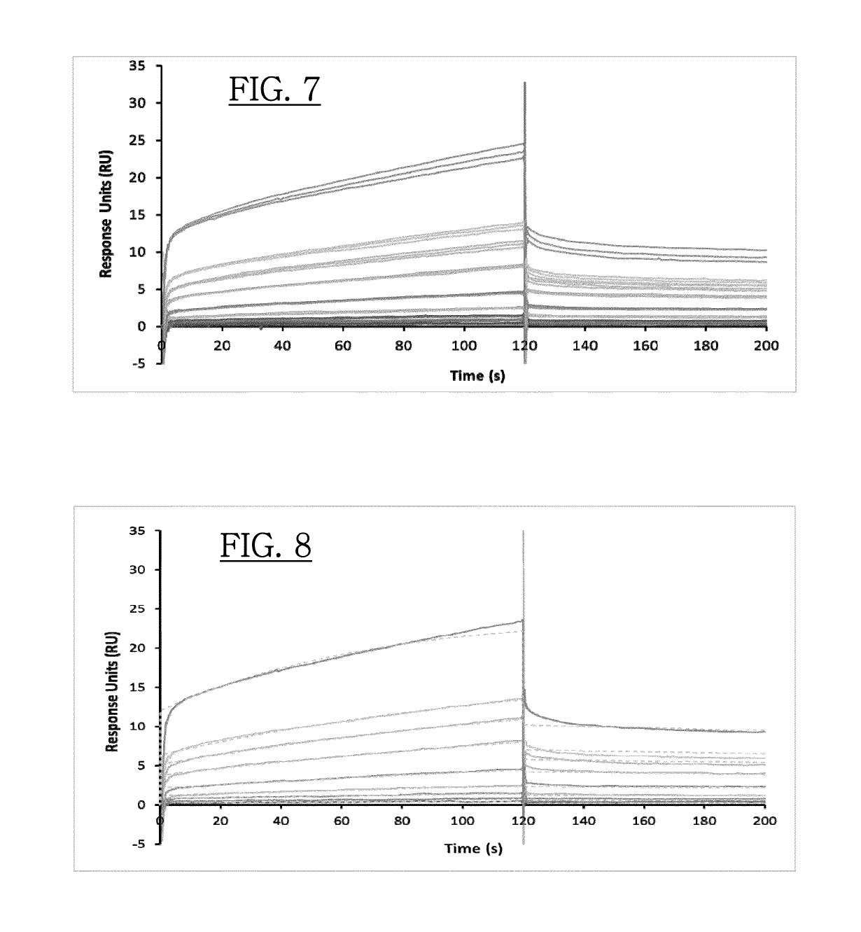

These results were corroborated by surface plasmon resonance experiments, the results of which are shown in FIGS. 7 and 8. In the SPR experiments, freshly prepared Aβ(1-40) was immobilized on a chip. Subsequently, several solutions containing different concentrations of B12 were passed over the immobilized Aβ(1-40). Referring to FIG. 7, a concentration dependent change in the refractive index at the surface of the chip was detected. This change, reflective of the Aβ peptide/B12 interaction, was monitored in real time. The the resulting sensorgrams of FIG. 7 were fit to obtain a binding constant of 131.5 μM. The results of the best-fit are shown in FIG. 8. Collectively, the fluorescence and SPR data demonstrated a direct interaction of B12 with the amyloid-beta peptide. The fact that the binding affinity is on a micromolar scale means that this effect is physiologically relevant.

### Example 2

### Vitamin B12 Dissociates Higher-Order Amyloid-Beta Peptide Forms and Inhibits Self-Association of Amyloid-Beta Peptide Monomers

This section shows that vitamin B12 actually dissociates amyloid-beta peptide monomer from higher-order amyloid-beta peptide structures such as oligomers and fibrils.

Thioflavin T Fluorescence Spectroscopy Experiments on Amyloid-Beta Peptide in the Presence of Vitamin B12.

The Fluorescence signal was measured as previously described using a Biotek Flx800 fluorescence microplate reader. Aβ(1-40) peptide samples (30 μM) were incubated at 37° C. in the presence or absence of B12. Following incubation, ThT was added to a final concentration of 10 μM as a probe for fibril formation by Aβ(1-40). All fluoresce readings were corrected for 10 mM sodium phosphate buffer contribution. Thioflavin T (ThT) (Sigma Aldrich) was reconstituted in Nanopure water, aliquoted, and stored at −20° C. Concentration of ThT was determined using UV-Vis spectroscopy (Cary300 spectrophotometer, Varian, Inc) and an extinction coefficient of 36,000 M⁻¹cm⁻¹ at 412 nm.

Results.

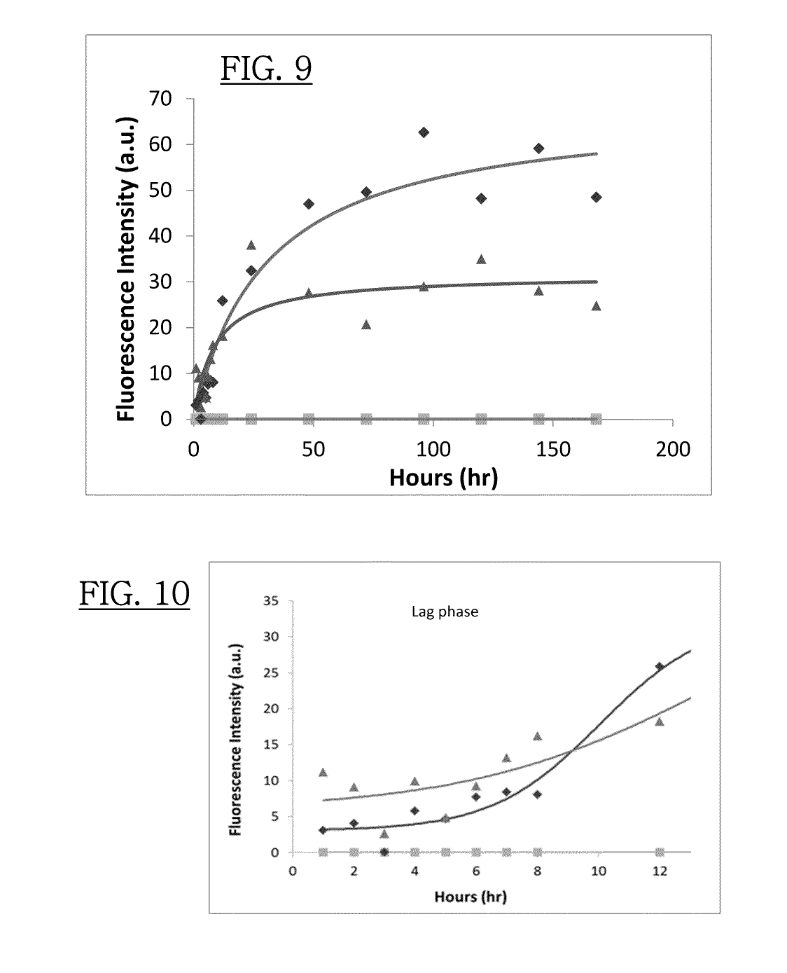

The formation of the higher order amyloid forms by the Aβ(1-40) peptide was monitored using Thioflavin T (“ThT”) fluorescence. These results are shown in FIGS. 9 and 10. In the absence of vitamin B12, the Aβ(1-40) formed higher-order aggregates, or fibrils, with a lag phase of approximately 8 hours. Addition of B12 to these aggregates resulted in a reduction of the ThT fluorescence, indicating fibril dissociation. Importantly, in the presence of vitamin B12, there was no increase in the ThT fluorescence signal, indicating that vitamin B12 inhibits self-association of the amyloid-beta peptide.

### Example 3

### Vitamin B12 Prevents Amyloid-Beta Peptide Induced Cell Death in Hippocampal Cells

This section shows that vitamin B12 protects hippocampal cells by preventing amyloid-beta peptide from forming toxic oligomers.

Neuronal Cell Culture Experiments.

Neuronal cell culture experiments were conducted using methodologies described previously by Lee, et al in Cell, Vol. 111(2), pages 219-230 (2002); Lei, et al in EMBO J, Vol. 21(12), pages 2977-2989 (2002); Xin, et al in J Neurosci, Vol. 25(1), pages 139-48 (2005); and Xin, et al in European Journal of Neuroscience, Vol. 21(3), pages 622-636 (2005).

Briefly, hippocampal tissue was dissected from Wistar rat embryos (18 days gestation or E18) at 4° C. Dissociated hippocampal neurons were plated at a density of approximately 2.0×10⁴ cells/cm² onto 35 mm culture dishes coated with poly-D-lysine. Neurons were matured in culture for 21 days in vitro in NEUROBASAL® media (Invitrogen) supplemented with B27 (2%), bFGF2 (2 ng/mL) and L-glutamine (0.5 mM). The Aβ(1-40) was dissolved in DMSO, followed by dilution into culture medium (final DMSO concentration was 1%), and incubation at 37° C. for three days to form Aβ(1-40) oligomeric species. This pre-aggregated Aβ was added to the culture medium at a final concentration of 25 μM on the 21^{st} day in vitro (DIV 21), a concentration that has been previously established to cause neurotoxicity without causing overt neuronal death in hippocampal cell culture. Some pre-aggregated Aβ samples were incubated with varying dosages of B12 (50, 25, or 12.5 μM final concentration). All cells were incubated for 72 hours. Cell culture medium containing 1% DMSO was used as a vehicle control to demonstrate that DMSO neither prevented nor accelerated neuronal cell death.

Trypan Blue Staining and Cell Counting.

Following the three-day incubation, Trypan Blue staining of dead neuronal cells was performed by removing 1 mL of medium from each dish and adding 50 μL of Trypan Blue (0.2%) (Harleco EMD). The dishes were gently mixed for two minutes prior to removal of all solution. Five random fields, one from each corner and one from the center of the dish, were counted for both live and dead cells at 63× magnification to obtain a percentage of cell death. Mann-Whitney U statistical analyses were performed using NCSS 2000 (NCSS, LLC) software. Following counting, a representative picture of each dish was taken.

The physiological relevance of the B12/Aβ interaction was evaluated using neuronal cell culture experiments. Because the hippocampus is the first region of the brain to be affected by the Alzheimer's pathology, these experiments focused specifically on this region. The amyloid-beta peptide was incubated for 3 days prior to addition to hippocampal cell culture to generate higher-order species demonstrated to be toxic by others.

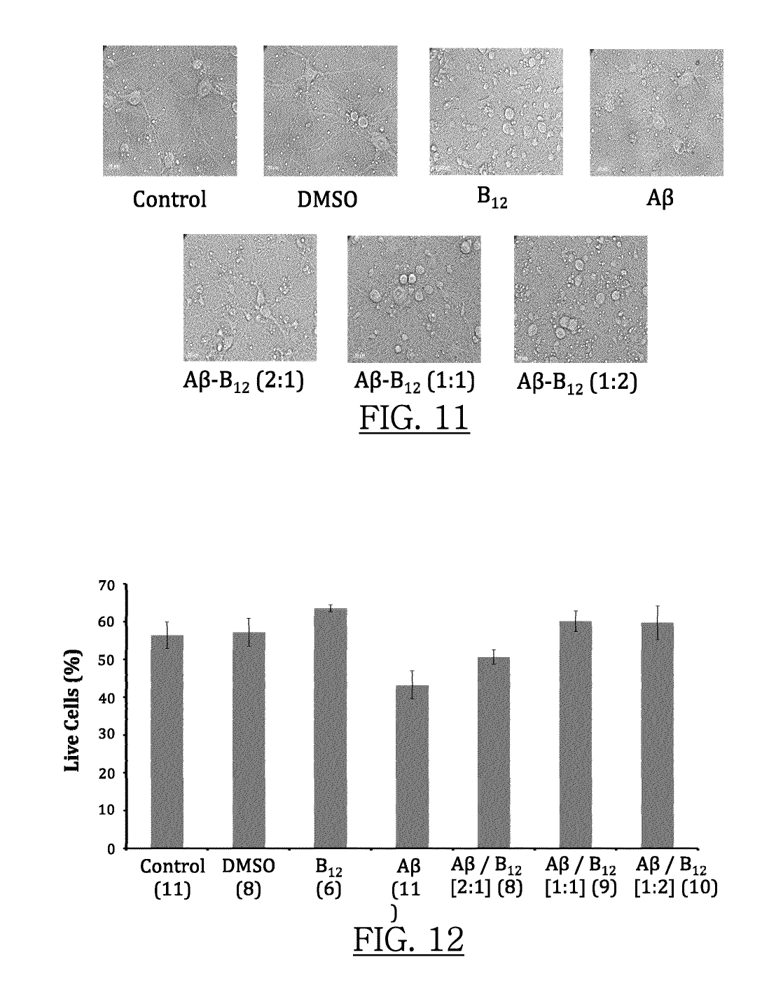

Referring to FIG. 11, micrographs . . . . It is notable, that (Addition of freshly prepared Aβ peptide to the hippocampal cell culture did not cause neuronal cell death; data not shown.) As depicted in FIG. 12, neuronal cell death is observed upon addition of the amyloid-beta peptide. Importantly, this effect is alleviated in the presence of B12, demonstrating its neuroprotective efficacy.

The invention has been described above with reference to the accompanying drawings, in which preferred embodiments of the invention are shown. Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described. The skilled should understand that the methods and materials used and described are examples and may not be the only ones suitable for use in the invention.

In the specification set forth above there have been disclosed typical preferred embodiments of the invention, and although specific terms are employed, the terms are used in a descriptive sense only and not for purposes of limitation. The invention has been described in some detail, but it will be apparent that various modifications and changes can be made within the spirit and scope of the invention as described in the foregoing specification and as defined in the appended claims.

That which is claimed is:

1. A method of treating amyloid-beta peptide aggregation in a patient having Alzheimer's disease, the method comprising:
determining a first level of amyloid-beta peptides in the patient;
establishing a first dose of vitamin B12 based on the first level of amyloid-beta peptides in the patient, the first dose being sufficient to promote dissociation of amyloid-beta peptide monomers from amyloid-beta peptide aggregates in the patient; and
administering the first dose to the patient.

2. The method of claim 1, wherein determining the first level of amyloid-beta peptides in the patient includes testing the patient's plasma for an amount of amyloid-beta peptides therein.

3. The method of claim 1, wherein the amount of vitamin B12 in the first dose is at least equimolar with a molarity of amyloid-beta peptides in the first level of amyloid-beta peptides.

4. The method of claim 1, further comprising:
determining a second level of amyloid-beta peptides in the patient after administering the first dose;
establishing a second dose of vitamin B12 based on the second level of amyloid-beta peptides in the patient; and
administering the second dose to the patient.

5. The method of claim 4, wherein the amount of vitamin B12 in the second dose is at least equimolar with a molarity of amyloid-beta peptides in the second level of amyloid-beta peptides.

6. A method of treating amyloid-beta peptide aggregation in a subject, the method comprising contacting at least one amyloid-beta peptide oligomer formed from a plurality of amyloid-beta peptide monomers with an effective amount of vitamin B12, the effective amount being sufficient to stimulate dissociation of the amyloid-beta peptide monomers from the amyloid-beta peptide oligomer.

7. The method of treating amyloid-beta peptide aggregation of claim 6, wherein the effective amount is a function of a quantity of amyloid-beta peptides in a sample taken from the subject.

8. The method of treating amyloid-beta peptide aggregation in a subject of claim 7, wherein the sample is a portion of the subject's plasma.

9. The method of treating amyloid-beta peptide aggregation in a subject of claim 7, wherein the effective amount is at least equimolar with a molarity of amyloid-beta peptides estimated to be in the subject based on the quantity of amyloid-beta peptides in the sample.

10. The method of treating amyloid-beta peptide aggregation in a subject of claim 6, wherein the effective amount is at least one molecule of vitamin B12 per amyloid-beta peptide monomer dissociated.

11. The method of treating amyloid-beta peptide aggregation in the subject of claim 6, wherein the subject is identified as having a condition characterized by amyloid-beta peptide aggregates.

12. A method of treating Alzheimer's disease comprising administering an effective amount of vitamin B12 to a patient, the effective amount being sufficient to cause dissociation of amyloid-beta peptide monomers from pre-existing amyloid-beta peptide oligomers in the patient, the effective amount being based on a pre-determined quantity of amyloid-beta peptide oligomers in the patient.

13. The method of treating Alzheimer's disease of claim 12, wherein the quantity of amyloid-beta peptide oligomers in the patient is estimated from a level of amyloid-beta peptides in a sample of the patient's plasma.

14. The method of treating Alzheimer's disease of claim 13, wherein the effective amount is at least an equimolar ratio of vitamin B12 to amyloid-beta peptides in the sample.

15. A method of preventing neuronal-cell death comprising:
estimating a level of amyloid-beta peptides in a subject;
establishing an effective amount of vitamin B12 based on the estimated level; and
administering the effective amount of vitamin B12 to the subject.

16. The method of preventing neuronal-cell death of claim 15, wherein estimating the level of amyloid-beta peptides in the subject includes testing the subject's plasma for a quantity of amyloid-beta peptides therein.

17. The method of preventing neuronal-cell death of claim 15, wherein the effective amount is at least an equimolar ratio of vitamin B12 to amyloid-beta peptides estimated to be in the subject.

18. A test for detecting the presence of amyloid-beta peptides, the test comprising:
stimulating a signal from a sample having been exposed to vitamin B12 and identified as being at risk for containing amyloid-beta peptides; and
detecting the signal, the signal being indicative of the presence or absence of amyloid-beta peptides.

19. The test of claim 18, wherein the signal is a spectroscopic signal.

20. The test of claim 19, wherein the spectroscopic signal is fluorescence from a fluorophore associated with with the sample.

21. The test of claim 20, further comprising determining a degree of aggregation of the amyloid-beta peptides by comparing successively detected fluorescence signals.

22. The test of claim 20, wherein the fluorophore is Thioflavin T.

23. The test of claim 19, wherein the spectroscopic signal is a surface plasmon resonance signal.

24. The test of claim 23, further comprising positioning the sample on a substrate and exposing the sample to vitamin B12 by flowing a vitamin B12 solution over the substrate.

25. The test of claim 23, further comprising determining a degree of binding between vitamin B12 and the amyloid-beta peptides by comparing successively detected surface plasmon resonance signals.
